(19) United States
(12) Patent Application Publication
(10) Pub. No.: US 2015/0157261 A1
(43) Pub. Date: Jun. 11, 2015

(54) SUBSTANCE DETECTION DEVICE AND WATCH-TYPE BODY FAT BURNING MEASUREMENT DEVICE

(71) Applicant: Seiko Epson Corporation, Tokyo (JP)

(72) Inventor: Yusuke Sakagami, Shiojiri (JP)

(73) Assignee: SEIKO EPSON CORPORATION, Tokyo (JP)

(21) Appl. No.: 14/411,825

(22) PCT Filed: Jun. 3, 2013
(86) PCT No.: PCT/JP2013/003472

§ 371 Date: Dec. 29, 2014

(30) Foreign Application Priority Data
Jun. 29, 2012 (JP) 2012-146529

(51) Int. Cl. A61B 5/00; G01N 21/65

(52) U.S. Cl. CPC A61B 5/4866; G01N 21/65; A61B 5/0075; A61B 5/742; A61B 5/681; G01N 2201/06113; G01N 2201/02

(57) ABSTRACT

A substance detection device capable of detecting a specific component in a biological gas collected from the skin is realized.

A substance detection device includes: a detection sample collection section which collects a biological gas released from the human skin, allows only this biological gas to pass through a permeable membrane, and stores the gas in a sensor chamber; a light source which excites a Raman scattered light from acetone in the collected biological gas; a sensor section which enhances the Raman scattered light by localized surface plasmon resonance; a spectrometer which disperses the enhanced Raman scattered light; a light receiving element; a signal processing and control circuit section which compares the acquired spectrum with the fingerprint spectrum of acetone which has been stored in advance and thereby identifies acetone, which is the collected substance to be detected, and calculates the amount of fat burning having a correlation with the concentration of acetone; and a display section.

### BACKGROUND

### Technical Field

The present invention relates to a substance detection device and a watch-type body fat burning measurement device.

### SUMMARY

Recently, as a method for improving lifestyle-related pathologies such as metabolic syndrome, regular aerobic exercise has been recommended, however, it is often the case that such exercise cannot be continued, and the effect of exercise cannot be enjoyed. If such a person can easily know the burning amount of body fat in order to increase the effect of exercise, the motivation to continue exercise is improved, and as a result, the effect of exercise can be expected to be enjoyed. Therefore, a device capable of measuring the amount of fat burning by exercise has been proposed. Therefore, a device in which the concentration of acetone (or the amount of acetone) in the expiration gas is detected, the exercise intensity is calculated by detecting a change in the concentration of acetone, and a fat burning ratio is calculated using the consumed amount of oxygen when loading the calculated exercise intensity, an energy per unit amount of oxygen consumed, and the ratio of consumed fat associated with this energy has been proposed (see, for example, JP-A-2010-268864).

On the other hand, a device for detecting a biological gas released from the skin and a method for monitoring the in vivo metabolic information based on the obtained detection information have been proposed (see, for example, JP-A-2010-148692).

In such JP-A-2010-268864, since a test subject wears a mask and the expiration gas is collected, it is necessary to stop exercise once to collect the expiration gas. Further, in the case where a skin gas is detected, the concentration of a biological gas to be detected is lower as compared with the expiration gas, and therefore, it is liable to be disturbed by water due to sweat or the like.

The invention has been made for solving at least part of the above problems, and can be realized as the following embodiments or application examples.

### APPLICATION EXAMPLE 1

A substance detection device according to this application example includes: a detection sample collection section which collects a biological gas released from the human skin, and stores the gas in a sensor chamber; a light source which excites a Raman scattered light from a substance to be detected in the collected biological gas; a sensor section which enhances the Raman scattered light by localized surface plasmon resonance; a spectrometer which disperses the enhanced Raman scattered light; a light receiving element which converts the dispersed light to an electrical signal and acquires the spectrum of the enhanced Raman scattered light; a signal processing and control circuit section which compares the acquired spectrum with the fingerprint spectrum of the substance to be detected which has been stored in advance and thereby identifies the collected substance to be detected, and calculates the concentration of the substance to be detected and the amount of a specific substance having a correlation with the concentration of the substance to be detected; and a display section which displays the results calculated by the signal processing and control circuit, wherein the detection sample collection section comes in close contact with the human skin, and the detection sample collection section includes a permeable membrane which allows the biological gas to pass through the sensor section.

This application example is configured such that a biological gas generated from the human skin is collected, a spectrum of a Raman scattered light by utilizing localized surface plasmon resonance generated by irradiating a sensor section with a light is compared with a fingerprint spectrum, thereby to identify a substance to be detected, and the amount of a specific substance having a correlation with the concentration (or amount) of the substance to be detected is calculated and displayed in the display section. Therefore, according to such a configuration, a substance detection device capable of detecting a trace amount of a substance to be detected contained in a biological gas with high sensitivity can be realized.

Further, the amount of a specific substance having a correlation with the concentration of the substance to be detected can be detected.

Further, although a detailed description will be made in the following embodiments, the substance detection device of this application example can reduce the size of the respective component elements constituting the device, and therefore, the size wearable on a test subject can be realized. Further, since a biological gas generated from the skin is collected, as compared with the above-described configuration in which the expiration gas is collected, the amount of a specific substance can be measured also during exercise.

Incidentally, if the volume of the sensor chamber is constant and the concentration of the substance to be detected is found, the amount (weight) of the substance to be detected can be determined.

On the other hand, in the biological gas, other than the substance to be detected, water is contained. If water is adhered to the sensor section, the Raman scattered light cannot be enhanced by localized surface plasmon resonance. Therefore, by using a permeable membrane which allows the biological gas as the substance to be detected to pass therethrough, but does not allow water to pass therethrough, the Raman scattered light can be enhanced by localized surface plasmon resonance with high efficiency.

### APPLICATION EXAMPLE 2

It is preferred that in the substance detection device according to the above application example, the sensor section includes a sensor chip having a metal nanostructure which is smaller than the wavelength of a light emitted from the light source.

In the case where a metal nanoparticle which is smaller than the wavelength of a light is irradiated with the light, in the vicinity of the metal nanoparticle, free electrons present on the light surface are resonated by the action of the electric field of the incident light, and electric dipoles are in an aligned state in the vicinity of the metal nanoparticle by the free electrons. Due to this, a stronger enhanced electric field than the electric field of the incident light is formed, and thus, localized surface plasmon resonance is generated. By the localized surface plasmon resonance, even in the case of a target molecule (a particle of the substance to be detected) present in a trace amount, Raman spectroscopy can be performed, and thus, a trace amount of the substance to be detected can be detected with high sensitivity.

### APPLICATION EXAMPLE 3

It is preferred that the substance detection device according to the above application example further includes a collected gas discharge unit which discharges the biological gas stored in the sensor chamber outside the sensor chamber.

If the collected biological gas is retained in the sensor chamber, in the subsequent detection of the substance to be detected, an accurate detection result cannot be obtained. Therefore, by discharging the biological gas outside the sensor chamber using the collected gas discharge unit before performing a redetection, an accurate detection result can be obtained.

### APPLICATION EXAMPLE 4

It is preferred that in the substance detection device according to the above application example, the detection sample collection section, the light source, the sensor section, the spectrometer, the light receiving element, the signal processing and control circuit section, and the display section are integrally housed so as to be wearable on the body.

According to this, for example, a substance detection device which is easy to carry like a watch type can be formed, and therefore, the device can be carried in daily life and also during exercise, and the detection result can be recognized by the display section.

### APPLICATION EXAMPLE 5

It is preferred that the substance detection device according to the above application example is divided into a main body section in which the detection sample collection section, the light source, the sensor section, and the display section are integrally housed, and a detection section in which the spectrometer, the light receiving element, and the signal processing and control circuit section are integrally housed, and the main body section and the detection section are connected to each other through an optical fiber which transmits the enhanced Raman scattered light and a cable which supplies electric power and transmits an electrical signal.

According to this configuration, the device is divided into the main body section and the detection section, and each section enables further reduction in size and weight as compared with the integrated device, and for example, the main body section can be attached to a wrist part where the display is easily visually recognized by the test subject oneself, and the detection section can be attached to an arbitrary place where the amount of exercise is small.

### APPLICATION EXAMPLE 6

It is preferred that in the substance detection device according to the above application example, the detection sample collection section is separated from a main body section in which the light source, the sensor section, and the display section are integrally housed, and the detection sample collection section and the sensor chamber are allowed to communicate with each other through a biological gas introduction tube.

According to this, since the detection sample collection section and the main body section are separated from each other, for example, when the main body section is attached to a wrist part, and the detection sample collection section is attached to an arm part in the vicinity of the main body section, an area from which the biological gas is collected by the detection sample collection section can be increased, and thus, the collection amount of the biological gas can be increased.

### APPLICATION EXAMPLE 7

It is preferred that the substance detection device according to the above application example includes a display section which is separated from a detection device main body section in which the detection sample collection section, the light source, the sensor section, the spectrometer, the light receiving element, and the signal processing and control circuit section are integrally housed, and the detection device main body section and the display section are connected to each other through a communication unit.

According to this configuration, the placement site of the display section is not limited, and the display section can be placed at an arbitrary site independent of the detection device main body section. The display section may be placed at a site apart from the test subject. In the case where the communication unit is a wireless communication unit, data detected by the detection device main body section is transmitted to, for example, a PC or a cellular phone, and the detection result can be displayed in the display section of such a device, and thus, the detection result can be recognized at a site apart from the test subject.

Further, by utilizing the memory of a PC or a cellular phone, the previous detection results and the cumulative values over a long period of time can be known.

### APPLICATION EXAMPLE 8

It is preferred that in the substance detection device according to the above application example, the substance to be detected is acetone, the specific substance is body fat, the signal processing and control circuit section calculates the burning amount of the body fat based on the amount of the detected acetone with reference to a non-protein respiratory quotient, and the display section displays the burning amount of the body fat.

Many free fatty acids produced in vivo are supplied to the liver, however, acetone which is a metabolite due to burning of fat in the liver accompanying exercise is released from the skin as a biological gas. Therefore, by detecting the concentration of acetone, it becomes possible to accurately measure the amount of fat burning. Accordingly, if the burning amount of body fat as the effect of exercise can be easily known by using the above-described substance detection device, the motivation to continue exercise of a test subject who is prone to metabolic syndrome is improved, and lifestyle-related pathologies can be improved.

### APPLICATION EXAMPLE 9

A watch-type body fat burning measurement device according to this application example includes: a display section provided on the outer surface of a watch-type case; a sensor section which detects a target substance in a biological gas released from a test subject by utilizing plasmon resonance; a light source section which irradiates the sensor section with a laser light to excite a Raman scattered light; a control section which calculates body fat burning according to the detected concentration of the target substance, and displays the calculation result in the display section; a close contact section which includes a permeable membrane for allowing the biological gas to pass therethrough and is capable of coming in close contact with a part of the arm of the test subject; and a wrist band which enables the close contact section to be attached to the arm of the test subject, wherein the display surface, the emitting direction of the laser light, and the permeable membrane are parallel to one another.

According to this configuration, the watch-type device can be made thin.

### BRIEF DESCRIPTION OF THE DRAWINGS

FIG. 1 shows a substance detection device according to a first embodiment, wherein (a) is a structural plan view seeing through the internal structure, (b) is a cross-sectional view showing a cross section taken along the line A-A in (a), and (c) is an external plan view.

FIG. 2 is a block diagram showing a main structure of the substance detection device according to the first embodiment.

FIG. 3 is an explanatory view schematically showing the principle of detection of a substance according to the first embodiment, wherein (a) is an explanatory view of Raman spectroscopy, (b) is an explanatory view of an enhanced electric field formed when a metal nanoparticle is irradiated with a light, and (c) is an explanatory view of surface enhanced Raman scattering in a metal nanostructure.

FIG. 4 is an explanatory view showing a relationship between fat burning and acetone, wherein (a) shows a flow from the intake of three major nutrients serving as major energy sources to the storage thereof, (b) shows the mechanism of fat burning, and (c) shows the time course of the utilization ratio of carbohydrate and fat in aerobic exercise.

FIG. 5 is a graph showing a relationship between the concentration of acetone and the signal intensity of acetone.

FIG. 6 is an explanatory view illustrating the attachment sites of an integrated substance detection device.

FIG. 7 shows a substance detection device according to a second embodiment, wherein (a) is an explanatory view of the overall structure, and (b) is a cross-sectional view of a main body section.

FIG. 8 is an external plan view of the main body section according to the second embodiment.

FIG. 9 shows a substance detection device according to a third embodiment, wherein (a) is an explanatory view of the overall structure, and (b) is a cross-sectional view showing a main body section.

FIG. 10 is an explanatory view of a structure of a substance detection device according to a fourth embodiment.

FIG. 11 shows relationships among an exercise intensity, a pulse rate, and the amount of fat burning, wherein (a) is a graph showing a relationship between an exercise intensity and the amount of fat burning, and (b) is a graph showing a relationship between a pulse rate and the amount of fat burning.

### DESCRIPTION OF EXEMPLARY EMBODIMENTS

Hereinafter, embodiments of the invention will be described by showing a substance detection device which detects the concentration of acetone contained in a biological gas and detects the burning amount of body fat having a correlation with the detected concentration of acetone as an example.

Incidentally, the drawings referred to in the following description are schematic views in which the vertical and horizontal scales of members or parts are different from the actual ones to make each of the members to have a recognizable size.

### First Embodiment

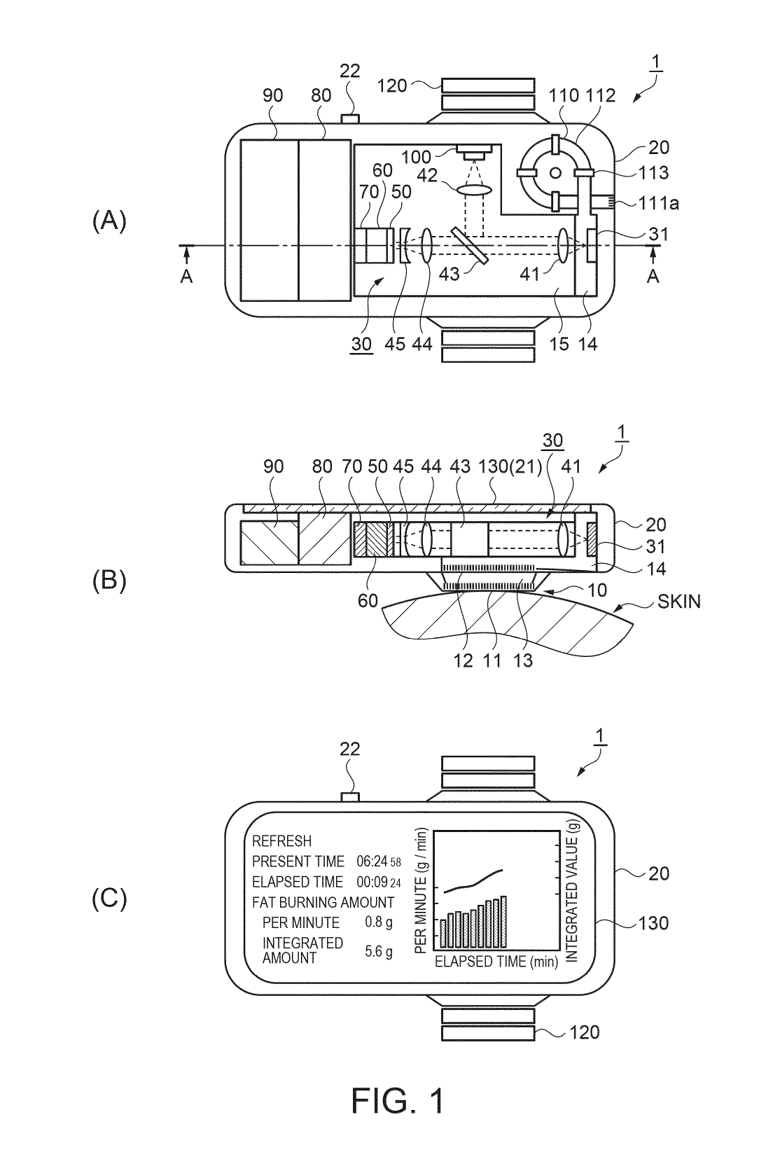

FIG. 1 shows a substance detection device 1 according to a first embodiment, wherein (a) is a structural plan view seeing through the internal structure, (b) is a cross-sectional view showing a cross section taken along the line A-A in (a), and (c) is an external plan view. In FIGS. 1(a) and 1(b), in the substance detection device 1, a detection sample collection section 10, a detection section 30, and a display section 130 are housed in a space formed by a case 20 and a windshield glass 21 (see FIG. 1(b)). The detection sample collection section 10 is placed on the side to come in contact with the human skin (on the rear side of the case 20), the detection section 30 is placed inside the case 20, and the display section 130 is placed at a site where it can be visually recognized by a test subject (on the front side of the case 20).

The detection sample collection section 10 includes a first permeable membrane 11 as a permeable membrane which comes in close contact with the human skin, and a second permeable membrane 12 which is placed with a space 13 from the first permeable membrane 11. The first permeable membrane 11 which comes in close contact with the human skin is formed from a membrane which has water repellency so that water such as sweat does not directly enter the detection section 30, and allows a biological gas generated from the skin (incidentally, the “biological gas” is sometimes referred to as “skin gas”) to pass therethrough. The first permeable membrane 11 is provided for preventing water and the like contained in the biological gas from adhering to a sensor section 31, which will be described later, when the biological gas is taken in the detection section 30.

The second permeable membrane 12 has the same function as that of the first permeable membrane 11, and is provided for further enhancing the above-described function of the first permeable membrane 11 by adopting a double-membrane structure with the first permeable membrane 11. Therefore, the formation of the permeable membrane into a double-membrane structure is not prerequisite, and the double-membrane structure can be selected according to a sweating amount or the like in a region of the body where the substance detection device 1 is attached.

The first permeable membrane 11 and the second permeable membrane 12 are placed on the human body side of the case 20 such that the first permeable membrane 11 comes in close contact with the skin by an attachment belt 120.

Incidentally, the substance detection device 1 shown in FIG. 1 illustrates a structure in the case where the device 1 is attached to a wrist part.

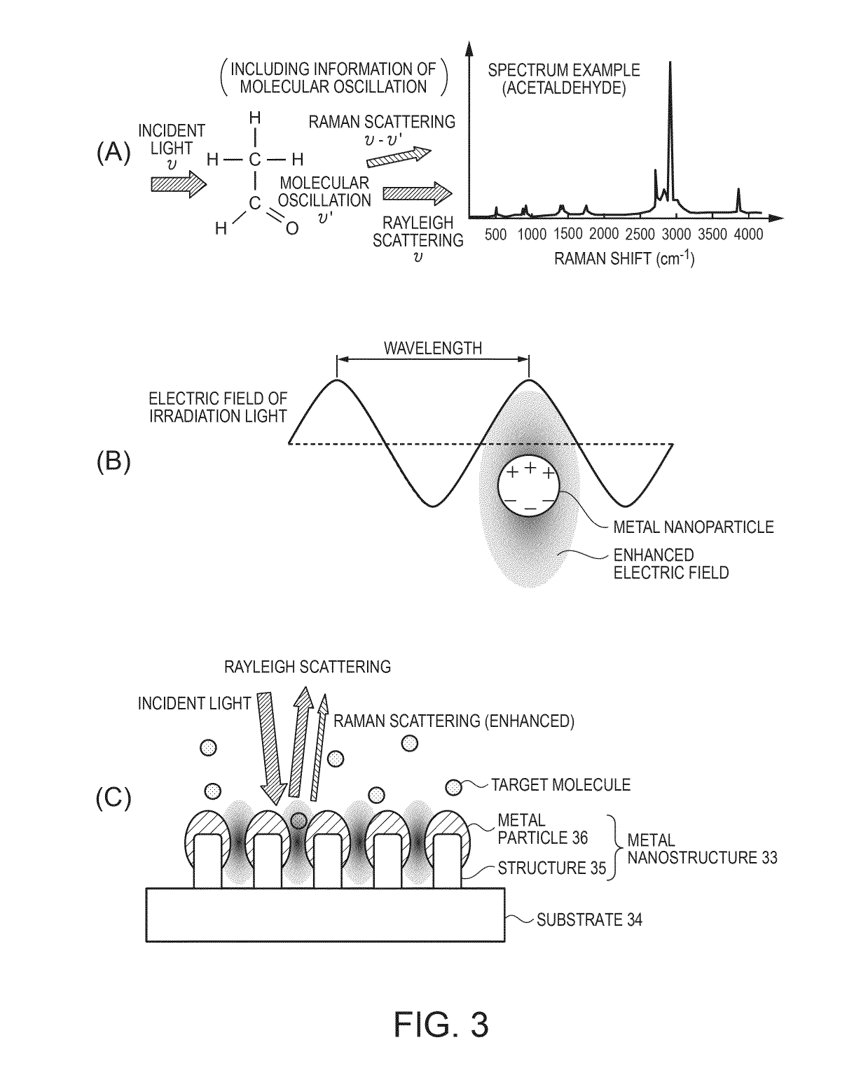

Next, the structure of the detection section 30 will be described. As shown in FIGS. 1(a) and 1(b), the detection section 30 is divided into a sensor chamber 14 and a detection chamber 15. The sensor chamber 14 is a space in which the biological gas released from the arm is stored, and the sensor section 31 is placed therein. The sensor section 31 includes a sensor chip which enhances a Raman scattered light. The structure and function of the sensor section 31 will be described later with reference to FIG. 3.

The detection chamber 15 includes a light source 100 which excites a molecule to be detected, a first lens group which condenses a light emitted from the light source 100 on the sensor section 31, and a second lens group which condenses a Raman scattered light having been enhanced (referred to as “enhanced Raman scattered light”) to be scattered from a sensor chip 32.

The first lens group is constituted by a lens 42 which converts the light emitted from the light source 100 into a parallel light, a half mirror 43 which reflects this parallel light toward the sensor section 31, and a lens 41 which condenses the light reflected by the half mirror 43 on the sensor section 31.

The second lens group is constituted by a lens 44 which condenses a Raman light enhanced by the sensor section 31 through the lens 41 and the half mirror 43, and a lens 45 which converts the condensed Raman light into a parallel light.

The detection chamber 15 further includes an optical filter 50 which removes a Rayleigh scattered light from the condensed scattered light, a spectrometer 60 which disperses the enhanced Raman scattered light into a spectrum, a light receiving element 70 which converts the dispersed spectrum to an electrical signal, a signal processing and control circuit section 80 which converts the dispersed spectrum to an electrical signal as information of a fingerprint spectrum specific to a substance detected from the biological gas, and an electric power supply section 90. The fingerprint spectrum has been stored in the signal processing and control circuit section 80 in advance.

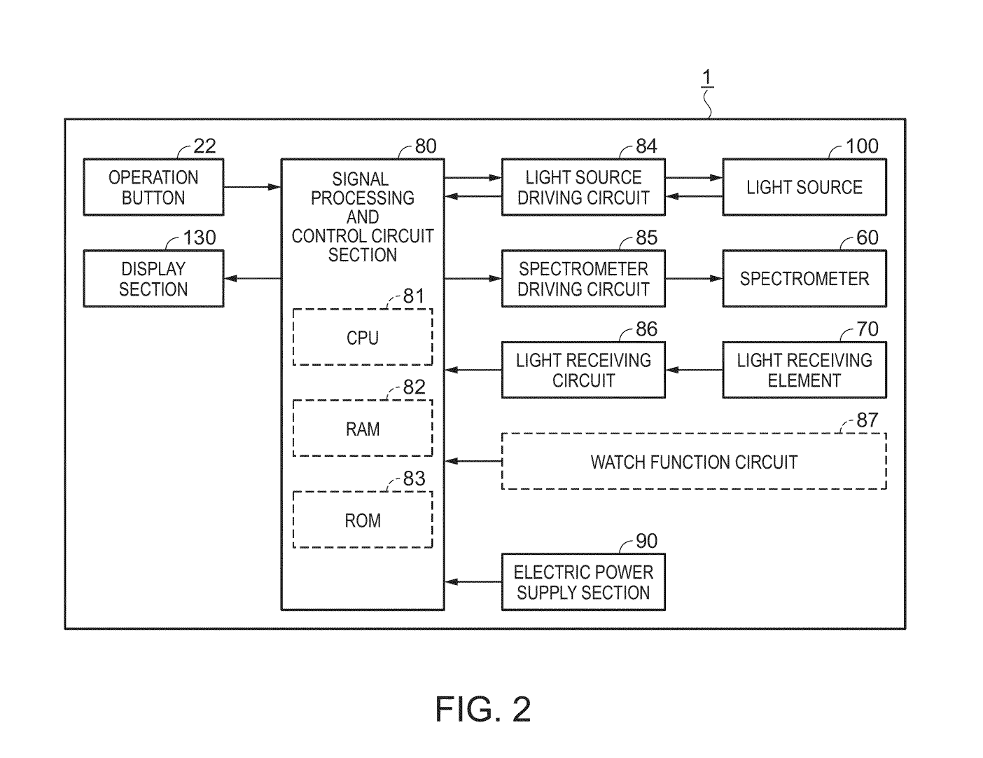

As the electric power supply section 90, a primary battery, a secondary battery, or the like can be used. In the case of a primary battery, with respect to the voltage which is decreased to a predetermined value or less, a CPU 81 compares the information stored in an ROM 83 (see FIG. 2 for both CPU 81 and ROM 83) with the obtained information of the voltage of the primary battery, and when the voltage of the primary battery is equal to or lower than a predetermined value, an indication of battery replacement is displayed in the display section 130.

In the case of a secondary battery, with respect to the voltage which is decreased to a predetermined value or less, a CPU 81 compares the information stored in an ROM 83 with the obtained information of the voltage of the secondary battery, and when the voltage of the secondary battery is equal to or lower than a predetermined value, an indication of recharging is displayed in the display section 130. A test subject sees the indication, connects a recharger to a connecting section (not shown), and recharges the battery until the voltage is increased to a predetermined value, whereby the battery can be repeatedly used.

Further, the substance detection device 1 of this embodiment includes a collected sample discharge unit 110 which discharges the biological gas collected in the sensor chamber 14 to the outside. The collected sample discharge unit 110 includes a discharge tube 112 with elasticity, in which one end thereof communicates with the sensor chamber 14, and the other end communicates with a discharge port 111a, and a plurality of rotating rollers 113. The collected sample discharge unit 110 is a so-called tube pump which is capable of discharging a gas in the sensor chamber 14 to the outside by pressing the discharge tube 112 from the sensor chamber 14 side to the discharge port 111a side by the rotating rollers 113.

The tube pump may be configured such that the rotation is performed by hand or the driving is performed by a motor. As the collected sample discharge unit, it is possible to appropriately select and use a gas discharge unit other than the tube pump.

The discharge port through which the biological gas is discharged from the sensor chamber 14 is more preferably configured such that the discharge port is provided at multiple sites for rapidly discharging the biological gas.

Next, with reference to FIG. 1(c), a description will be made by showing one example of the display contents of the display section 130. The display section 130 uses an electrooptical display device such as a liquid crystal display device. As shown in FIG. 1(c), examples of the main display contents include a present time, an elapsed time from the start of measurement, a burning amount per minute and an integrated value thereof as the amount of fat burning, and a graph display showing the transition thereof. Further, after the measurement of the amount of fat burning, it is necessary to remove the gas in the sensor chamber 14 (that is, to refresh the sensor chip 32), and an indication for informing the operator of the same is also included. For example, in the case where “refresh” is displayed, an operation of discharging the collected sample is performed.

Further, although it is not shown in the drawing, according to the voltage of the electric power supply section 90, an indication of battery replacement or an indication of recharging is displayed.

Further, a watch function such as a time or a calendar may be displayed on demand.

Incidentally, in the case 20, an operating section 22 is placed, and operations such as start of detection, end of detection, and reset are performed.

The principle of the detection of the amount of fat burning will be described later with reference to FIGS. 3, 4, and 5.

Next, the structure and function of the substance detection device 1 including a control system will be described with reference to FIG. 2.

FIG. 2 is a block diagram showing a main structure of the substance detection device 1 according to this embodiment. The substance detection device 1 includes the signal processing and control circuit section 80 which controls the entire control system, and the signal processing and control circuit section 80 includes a CPU (Central Processing Unit) 81, an RAM (Random Access Memory) 82, and an ROM (Read Only Memory) 83.

In the above-described sensor chamber 14, a sensor chip and a sensor detector (not shown) for detecting the presence or absence of the sensor chip and reading a code are provided, and the information thereof is sent to the CPU 81 through a sensor detection circuit. A state where such information has been input is a state where the detection can be started, and therefore, the information that the device is ready for operation is input from the CPU 81 to the display section 130 and displayed in the display section 130.

When the CPU 81 receives a signal of start of detection from the operation section 22, alight source operating signal is output from a light source driving circuit 84, and the light source 100 is operated. In the light source 100, a temperature sensor and a light amount sensor are incorporated, and therefore, it can be confirmed that the light source 100 is in a stable state. When the light source 100 is stabilized, a biological gas is collected in the sensor chamber 14. Incidentally, in the collection of the biological gas, a suction pump (not shown) may be used.

The light source 100 is a laser light source which emits a stable linearly polarized light with a single wavelength, and is driven by a light source driving circuit 84 based on a signal from the CPU 81 and emits a light. The sensor chip 32 is irradiated with this light through the lens 42, the half mirror 43, and the lens 41, and a Rayleigh light and a Raman scattered light enhanced by an enhanced electric field (SERS: surface enhanced Raman scattering) enter the light receiving element 70 through the lens 41, the half mirror 43, the lens 44, the lens 45, the optical filter 50, and the spectrometer 60. The spectrometer 60 is controlled by a spectrometer driving circuit 85. Further, the light receiving element 70 is controlled by a light receiving circuit 86.

The optical filter 50 (see FIGS. 1(a) and 1(b)) blocks the Rayleigh light, and only the SERS (surface enhanced Raman scattering) light enters the spectrometer 60. In the case where as the spectrometer 60, a wavelength variable etalon utilizing Fabry-Perot resonance is adopted, the band width (λ1 to λ2) and the half width of a transmitting light have already been set, and by sequentially changing the wavelength of the transmitting light by an increment of the half width from λ1, the intensity of a light signal with the half width is converted to an electrical signal by the light receiving element 70 repeatedly until λ2. By doing this, the spectrum of the detected SERS light is obtained.

The thus obtained spectrum of the SERS light from the substance to be detected (here, acetone) is compared with a fingerprint spectrum stored in the ROM 83 of the signal processing and control circuit section 80, the target substance is identified, and the concentration of acetone is detected. Then, based on the concentration of acetone, the amount of fat burning is calculated. In order to inform a test subject of the calculation result, the result information is displayed in the display section 130 from the CPU 81. One example of the result information is shown in FIG. 1(c).

The watch function for measuring the measurement time displays the present time based on a preset time and the start time and the end time of measurement of fat burning by receiving the signal of the start of measurement of fat burning by means of a well-known watch function circuit 87. Further, the device has a watch function for displaying the amount of fat burning per minute, an integrated amount from the start of measurement of fat burning, and the like.

Next, the principle of detection of a substance of this embodiment will be described.

FIG. 3 is an explanatory view schematically showing the principle of detection of a substance in this embodiment, wherein (a) is an explanatory view of Raman spectroscopy, (b) is an explanatory view of an enhanced electric field formed when a metal nanoparticle is irradiated with a light, and (c) is an explanatory view of surface enhanced Raman scattering in a metal nanostructure.

First, Raman spectroscopy will be described with reference to FIG. 3(a). When a target molecule (a molecule of a substance to be detected) is irradiated with an incident light (wavelength: ν), most of the incident light is scattered as a Rayleigh scattered light without changing the wavelength. Part of the incident light is scattered as a Raman scattered light (wavelength: ν-ν′) including the information of the molecular oscillation of the target molecule. From the Raman scattered light, the fingerprint spectrum of the target molecule (here, acetaldehyde is shown as an example) is obtained. By this fingerprint spectrum, it is possible to identify the detected substance as acetaldehyde. However, the intensity of the Raman scattered light is very low, and therefore, it was difficult to detect a substance which is present only in a trace amount.

Therefore, with reference to FIG. 3(b), an enhanced electric field formed when a metal nanoparticle which is smaller than the wavelength of an incident light is irradiated with a light will be described. In the case where a metal nanoparticle is irradiated with a light, free electrons present on the surface of the metal nanoparticle are resonated by the action of the electric field of the incident light, and electric dipoles are in an aligned state in the vicinity of the metal nanoparticle by the free electrons. Due to this, a stronger enhanced electric field than the electric field of the incident light is formed. This phenomenon is a phenomenon specific to a metal particle which is smaller than the wavelength of a light and is called “localized surface plasmon resonance”.

Next, surface enhanced Raman scattering in a metal nanostructure will be described with reference to FIG. 3(c). The sensor chip 32 in this embodiment includes this metal nanostructure 33.

The metal nanostructure 33 is configured such that a metal nanoparticle 36 is formed at a tip end of each of columnar structures 35 arranged in a matrix on a substrate 34.

A phenomenon in which when a Raman scattered light is generated in an enhanced electric field, the Raman scattered light is enhanced by the effect of the enhanced electric field is surface enhanced Raman scattering (SERS). As shown in FIG. 3 (c), the metal nanostructures 33 are formed on the substrate 34 and are arranged so that an enhanced electric field is formed in a gap therebetween. When a target molecule enters, the Raman scattered light from the target molecule is enhanced by the enhanced electric field, and thus, a strong Raman signal is obtained. As a result, even if the target molecule is present in a trace amount, Raman spectroscopy can be performed. Due to this, a trace amount of a target molecule (detection target substance) can be detected with high sensitivity.

The substance detection device 1 of this embodiment is a device capable of detecting a component contained in a biological gas, and by detecting acetone in the biological gas, how much fat is burned can be known. For example, as a recent method for improving lifestyle-related pathologies such as metabolic syndrome, regular aerobic exercise has been recommended, and by making the amount of fat burning as the effect of exercise be able to be easily known, it is possible to improve the lifestyle-related pathologies.

Therefore, a relationship between fat burning and detection of acetone will be described.

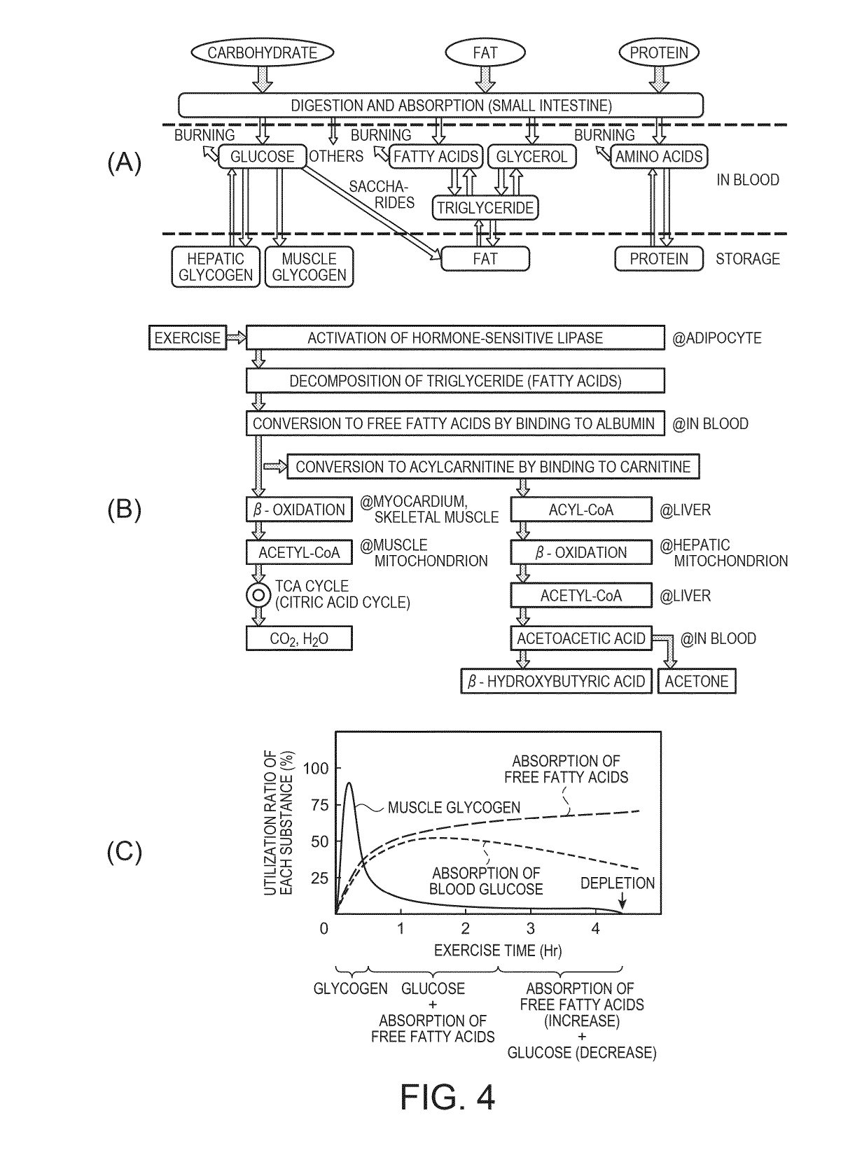

FIG. 4 is an explanatory view showing a relationship between fat burning and acetone, wherein (a) shows a flow from the intake of three major nutrients serving as major energy sources to the storage thereof, (b) shows the mechanism of fat burning, and (c) shows the time course of the utilization ratio of carbohydrate and fat in aerobic exercise.

As shown in FIG. 4(a), carbohydrate, fat, and protein, which are three major nutrients taken in from the diet, are digested in the stomach, and further digested in the small intestine, and then absorbed there. The absorbed nutrients are converted as follows, respectively, and circulate in the blood: carbohydrate is converted to glucose; fat is converted to fatty acids and glycerol; and protein is converted to amino acids. Some are burned, and the rest are converted to as follows and stored, respectively: glucose is converted to hepatic glycogen or muscle glycogen; fatty acids and glycerol are converted to fat via triglyceride; and amino acids are converted to protein, and according to need, they undergo the reverse process and are consumed. The energy per unit weight of each of these three major nutrients when it is burned corresponds to 4 kcal/g in the case of carbohydrate, 9 kcal/g in the case of fat, and 4 kcal/g in the case of protein (practical food calories). However, fat contains water when it is stored in white adipocytes, and therefore, the energy per unit weight of fat corresponds to 7.2 kcal/g.

As shown in FIG. 4(b), the mechanism of fat burning is as follows. When exercise is performed, adrenaline is released to activate a hormone-sensitive lipase in adipocytes, and the decomposition of triglyceride is promoted, and therefore, triglyceride is converted to fatty acids and glycerol. Since fatty acids cannot circulate in the blood as such, the fatty acids are bound to albumin and converted to free fatty acids, thereby circulate in the blood. Some are supplied to the myocardium or the skeletal muscle and decomposed by β-oxidation into acetyl-CoA while generating NADH₂⁺ and FADH₂. Then, acetyl-CoA undergoes the TCA cycle (commonly known as “citric acid cycle”), thereby generating ATP (Adenosine Triphosphate), and is converted to carbon dioxide (CO₂) and water (H₂O) in the end.

In the skeletal muscle, glycogen is mainly consumed as energy, and free fatty acids are consumed less. In the myocardium, about 70% of the total amount of energy is consumed as free fatty acids.

On the other hand, many of the free fatty acids are bound to carnitine and converted to acylcarnitine, which is supplied to the liver. In the liver, the acylcarnitine is converted to acyl-CoA and subjected to β-oxidation in hepatic mitochondria, whereby acetyl-CoA is formed. Further, acetyl-CoA is converted to acetoacetic acid, and further converted to β-hydroxybutyric acid and acetone. Acetoacetic acid, β-hydroxybutyric acid, and acetone are collectively referred to as “ketone body”, and only acetone is transformed into a gas and circulates in the blood and released as a component of an expiration gas or a skin gas. When viewed from a fat burning ratio, the ratio in the liver is higher than in the skeletal muscle or the heart, and the fat burning and acetone are correlated with each other. Therefore, by measuring the amount of acetone in the expiration gas or the amount of acetone in the skin gas, the amount of fat burning can be known.

Next, the time course of the utilization ratio of carbohydrate and fat in aerobic exercise will be described with reference to FIG. 4(c).

Step 1: ATP is synthesized by the metabolism of muscle glycogen.

Step 2: Accompanying a decrease in muscle glycogen, utilization of blood glucose is started, and fat in adipose tissues is released in the blood as free fatty acids. Then, by using blood glucose and free fatty acids as fuels, ATP is synthesized by oxidative metabolism. It is said that fat burning becomes active after 15 to 20 minutes from the start of exercise. It is not that active fat burning occurs at any exercise intensity, but fat burning becomes active at a relatively low exercise intensity. When the exercise intensity is increased, the exercise becomes anaerobic, and therefore, the amount of fat burning decreases and glycogen is mainly consumed instead.

As described above, by detecting the concentration of acetone in the human skin gas, how much fat is burned can be known. Therefore, a relationship between the concentration of acetone and the signal intensity of acetone will be described.

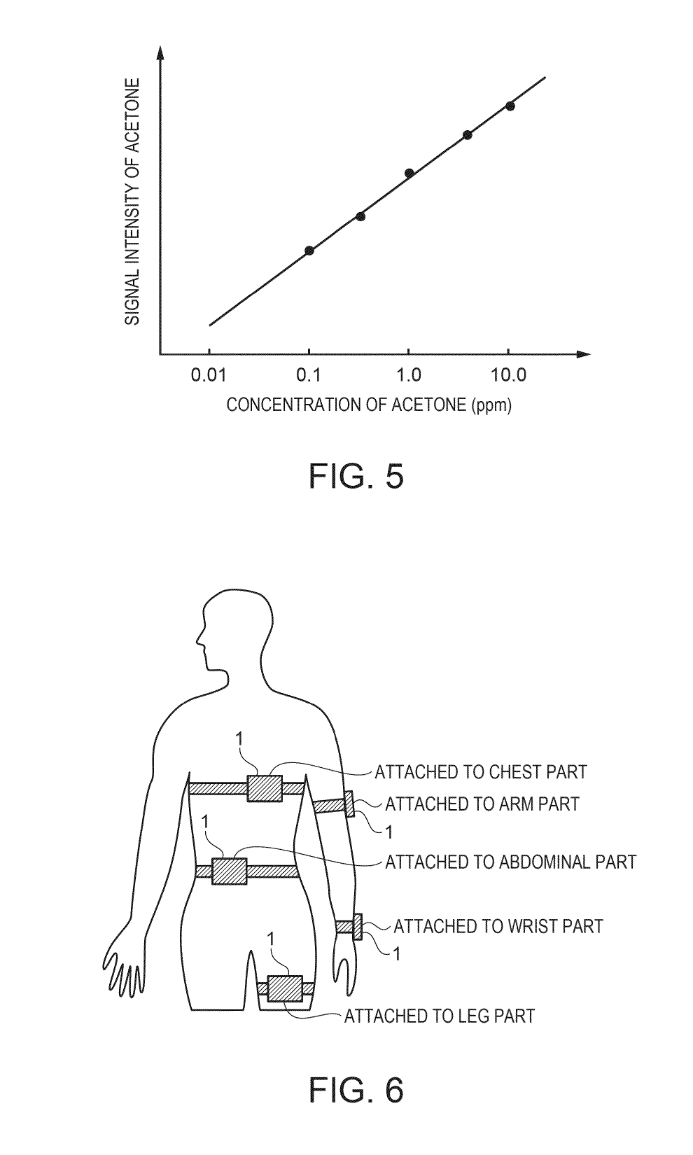

FIG. 5 is a graph showing a relationship between the concentration of acetone and the signal intensity of acetone. Incidentally, FIG. 5 is a graph showing a correlation between the concentration of acetone and the signal intensity of acetone, which is created as follows. Sample gasses having a different acetone concentration are adjusted and prepared, and detection of acetone is performed for the respective samples. The signal intensity of acetone is determined for a particularly strong peak among the respective spectra of acetone. As shown in FIG. 5, the concentration of acetone (expressed as an exponent) and the signal intensity of acetone can be represented by a substantially straight line.

Incidentally, the concentration of acetone can be replaced by the amount of acetone if the volume of the sensor chamber 14 is known.

Next, based on the detected concentration of acetone (amount of acetone), the amount of fat burning is calculated.

As described above, the three major nutrients are carbohydrate, fat, and protein, and the constituent ratio of carbon atoms, oxygen atoms, hydrogen atoms, and the like is different from one another. Therefore, during internal respiration, the ratio of consumed O₂ to produced CO₂ is different according to which nutrient is decomposed. When a given nutrient is mainly metabolized in somatic cells as a whole, the respiration also reflects the ratio. It is “respiratory quotient RQ” that expresses the ratio, and is represented by the following formula.

Respiratory quotient RQ=(discharged amount of CO₂ per unit time)/(consumed amount of O₂ per unit time)

There are very few oxygen atoms in fatty acids per se, and therefore, when fat is decomposed, a lot of oxygen has to be consumed. Since the produced amount of CO₂ is small for the consumed amount of O₂, fat has a respiratory quotient of 0.70, which is the lowest of the three major nutrients. Fat has a low oxygen content and has an energy value per unit weight of 9.3 kcal/g, which is the highest of the three major nutrients. Fat is a nutrient suitable in the case where energy is stored, and it is also fat that is stored under the skin due to overeating.

Carbohydrate generally has an atomic ratio as follows: C₆H₁₂O₆. Since a lot of oxygen atoms are contained, carbohydrate can be decomposed even if the consumed amount of oxygen is small. Carbohydrate has a respiratory quotient of 1.00, which is the highest of the three major nutrients. On the other hand, since the content of oxygen is high, the energy value per unit weight is 4.1 kcal/g, which is the lowest of the three major nutrients.

Protein has an atomic ratio between those of fat and carbohydrate and has a respiratory quotient of 0.85 and an energy value of 5.3 kcal/g. Theoretically, the respiratory quotient RQ can be 9 or higher, however, clinically, it hardly exceeds 1. On the other hand, when the respiratory quotient RQ is 0.7, it is indicated that fat utilization occurs, and when the respiratory quotient RQ is 0.7 or less, it is a fasting state and the production of a ketone body (ketosis) occurs. Very recently, it can be considered that the respiratory quotient RQ is constant in a resting state and the variation in respiratory quotient RQ in an individual is known to be within the range of 0.78 to 0.87.

The energy generated when each of the three major nutrients and a ketone body is oxidized is represented by the following formula.

(1) In the case where carbohydrate is oxidized

C₆H₁₂O₆+6O₂→6CO₂+6H₂O+36ATP(657 kcal)

[RQ=6CO₂/6O₂=1.0]

(2) In the case where fat is oxidized

C₅₅H₁₀₂O₆+77.5O₂→55CO₂+51H₂+429ATP (7,833 kcal)

[RQ=55CO₂/77.5O₂=0.71]

(3) In the case where protein is oxidized

C₁₀₀H₁₅₉O₃₂S_{0.7}+105.3O₂→13CON₂H₄(urea)+87CO₂+52.8H₂O+0.7H₂SO₄+27ATP (4,948 kcal)

[RQ=87CO₂/105.3O₂=0.83]

(4) In the case where a ketone body is produced from fat

0.176 g (fat)+0.437LO₂→1 g (ketone body)+0.11LCO₂+0.129H₂O+2,039 kcal

[RQ=0.111LCO₂/0.437LO₂=0.25]

Fat burning ratio (g/min)=consumed amount of oxygen (L/min)×energy per liter of oxygen (kcal/L)×fat burning ratio (%)×weight corresponding to energy value of fat (g/kcal)

Here, the “consumed amount of oxygen (L/min)” is a value measured by an expiration gas analyzer, and the “energy per liter of oxygen (kcal/L)” is calculated by conversion to an energy per liter of oxygen (kcal/L) shown in Table 1 based on a respiratory quotient (RQ) value measured by an expiration gas analyzer.

TABLE 1
Table for obtaining burning ratio of carbohydrate
and fat and generated energy based on non-protein
respiratory quotient (prepared by Lusk in 1924)

| | Burning ratio (%) | | Generated energy |
|---|---|---|---|
| RQ | Carbohydrate | Fat | (kcal/L of O₂) |
| 0.707 | 0.0 | 100.0 | 4.686 |
| 0.71 | 1.1 | 98.9 | 4.690 |
| 0.72 | 4.8 | 95.2 | 4.702 |
| 0.73 | 8.4 | 91.6 | 4.717 |
| 0.74 | 12.0 | 88.0 | 4.727 |
| 0.75 | 15.0 | 84.4 | 4.730 |
| 0.76 | 19.2 | 80.9 | 4.751 |
| 0.77 | 22.8 | 77.2 | 4.764 |
| 0.78 | 26.8 | 73.7 | 4.776 |
| 0.79 | 29.9 | 70.1 | 4.788 |
| 0.80 | 33.4 | 66.6 | 4.801 |
| 0.81 | 36.9 | 63.1 | 4.813 |
| 0.82 | 40.3 | 59.7 | 4.825 |
| 0.83 | 43.8 | 56.2 | 4.838 |
| 0.84 | 47.2 | 52.8 | 4.850 |
| 0.85 | 50.7 | 49.3 | 4.862 |
| 0.86 | 54.1 | 45.9 | 4.875 |
| 0.87 | 57.5 | 42.5 | 4.887 |
| 0.88 | 60.8 | 39.2 | 4.899 |
| 0.89 | 64.2 | 35.8 | 4.911 |
| 0.90 | 67.5 | 32.5 | 4.924 |
| 0.91 | 70.8 | 29.2 | 4.936 |
| 0.92 | 74.1 | 26.9 | 4.948 |
| 0.93 | 77.4 | 22.6 | 4.961 |
| 0.94 | 80.7 | 19.3 | 4.973 |
| 0.95 | 84.0 | 16.0 | 4.985 |
| 0.96 | 87.2 | 12.8 | 4.998 |
| 0.97 | 90.4 | 9.6 | 5.010 |
| 0.98 | 93.6 | 6.4 | 5.022 |
| 0.99 | 96.8 | 3.2 | 5.035 |
| 1.00 | 100.0 | 0.0 | 5.047 |

Table 1 is a table for obtaining a burning ratio of carbohydrate and fat and a generated energy based on a non-protein respiratory quotient. A fat burning ratio (%) can be represented by the ratio of carbohydrate and fat in burning with respect to a respiratory quotient in Table 1 and the weight corresponding to the energy of fat is 0.1097 (g/kcal) because an energy of 7,833 kcal is generated when fat C₅₅H₁₀₂O₆ (859.395 g/mol) is burned.

The correlation between the thus obtained fat burning ratio and the amount of acetone released from the skin per minute has been measured and compared in advance and a fat burning ratio can be calculated based on the measured amount of acetone released from the skin per minute.

The substance detection device 1 according to this embodiment described above has an integrated structure in which the detection sample collection section 10, the detection section 30, and the display section 130 are housed in the case 20. The substance detection device 1 having such a structure can be attached to various sites of the body with the attachment belt 120. The examples of the attachment site are shown in FIG. 6.

FIG. 6 is an explanatory view illustrating the attachment sites of the integrated substance detection device 1. As shown in FIG. 6, the substance detection device 1 can be attached to a wrist part, an arm part, a chest part, a waist part, a leg part, and the like. At this time, the attachment site is not particularly limited as long as the device can be attached so that the detection sample collection section 10 comes in close contact with the skin. However, if the device is attached to a wrist part, the attachment feeling is similar to the case where a watch is attached, and moreover, the display section 130 is easily visually recognized by a test subject (a person who wears the device) oneself in a state where the substance detection device 1 is attached. Therefore, the amount of fat burning can be recognized at any time, and thus, the device is highly convenient.

The substance detection device 1 according to this embodiment is configured such that a biological gas generated from the human skin is collected, a spectrum of a Raman scattered light by utilizing localized surface plasmon resonance generated by irradiating the sensor section 31 with a light is compared with a fingerprint spectrum, thereby to identify the substance to be detected, and the amount of a specific substance having a correlation with the concentration (or amount) of the substance to be detected is calculated and displayed in the display section 130. Therefore, according to such a configuration, the substance detection device 1 capable of detecting a trace amount of a substance to be detected contained in a biological gas with high sensitivity can be realized.

The substance to be detected exemplified in this embodiment is acetone, and the specific substance is body fat. Therefore, by detecting the concentration of acetone, an accurate amount of fat burning can be determined. Accordingly, if the burning amount of body fat as the effect of exercise can be easily known by using the above-described substance detection device 1, the motivation to continue exercise of a test subject who is prone to metabolic syndrome is improved, and lifestyle-related pathologies can be improved.

Further, the substance detection device 1 according to this embodiment can reduce the size of the respective component elements constituting the device, and therefore, the size wearable on a test subject can be realized. Further, a biological gas generated from the skin is collected, and therefore, unlike the above-described configuration in which the expiration gas is collected, the amount of fat burning can be measured also during exercise.

Further, the detection sample collection section 10 includes the first permeable membrane 11 which comes in close contact with the human skin and allows a biological gas to pass through the sensor section 31, and the second permeable membrane 12. In the biological gas, water is contained other than acetone. If water is adhered to the sensor section 31, the Raman scattered light cannot be enhanced by localized surface plasmon resonance. Therefore, by using the permeable membrane which allows the biological gas to pass therethrough, but does not allow water to pass therethrough, the Raman scattered light can be enhanced by localized surface plasmon resonance with high efficiency.

Further, the sensor section 31 includes the sensor chip 32 having the metal nanostructure 33 which is smaller than the wavelength of a light emitted from the light source 100. Since the metal nanostructure 33 is used in this manner, by the localized surface plasmon resonance, even in the case of a target molecule (acetone molecule) present in a trace amount, Raman spectroscopy can be performed, and thus, a trace amount of acetone can be detected with high sensitivity.

Further, the substance detection device 1 according to this embodiment further includes the collected sample discharge unit 110 which discharges the biological gas stored in the sensor chamber 14 outside the sensor chamber 14. If the collected biological gas is retained in the sensor chamber 14, in the subsequent detection operation, an accurate detection result is not obtained. Therefore, by discharging the biological gas outside the sensor chamber 14 using the collected sample discharge unit 110 before performing a detection operation again, an accurate detection result can be obtained.

As shown in FIG. 1, the substance detection device 1 according to this embodiment constitutes a watch-type body fat burning measurement device, and therefore can be carried in daily life and also during exercise, and thus has an effect that the amount of fat burning as the result of exercise can be recognized there by the test subject oneself.

### Second Embodiment

Next, a substance detection device 2 according to a second embodiment will be described. The substance detection device 1 according to the first embodiment described above has a watch-type structure in which the detection sample collection section 10, the detection section 30, and the display section 130 are integrated. On the other hand, the second embodiment has a characteristic that the device is divided into a main body section 200 in which a detection sample collection section 10, a light source 100, a sensor section 31, and a display section 130 are integrated, and a detection section 250 in which a spectrometer 60, a light receiving element 70, and a signal processing and control circuit section 80 are integrated, and the main body section 200 and the detection section 250 are connected to each other through an optical fiber 210 and a cable 220.

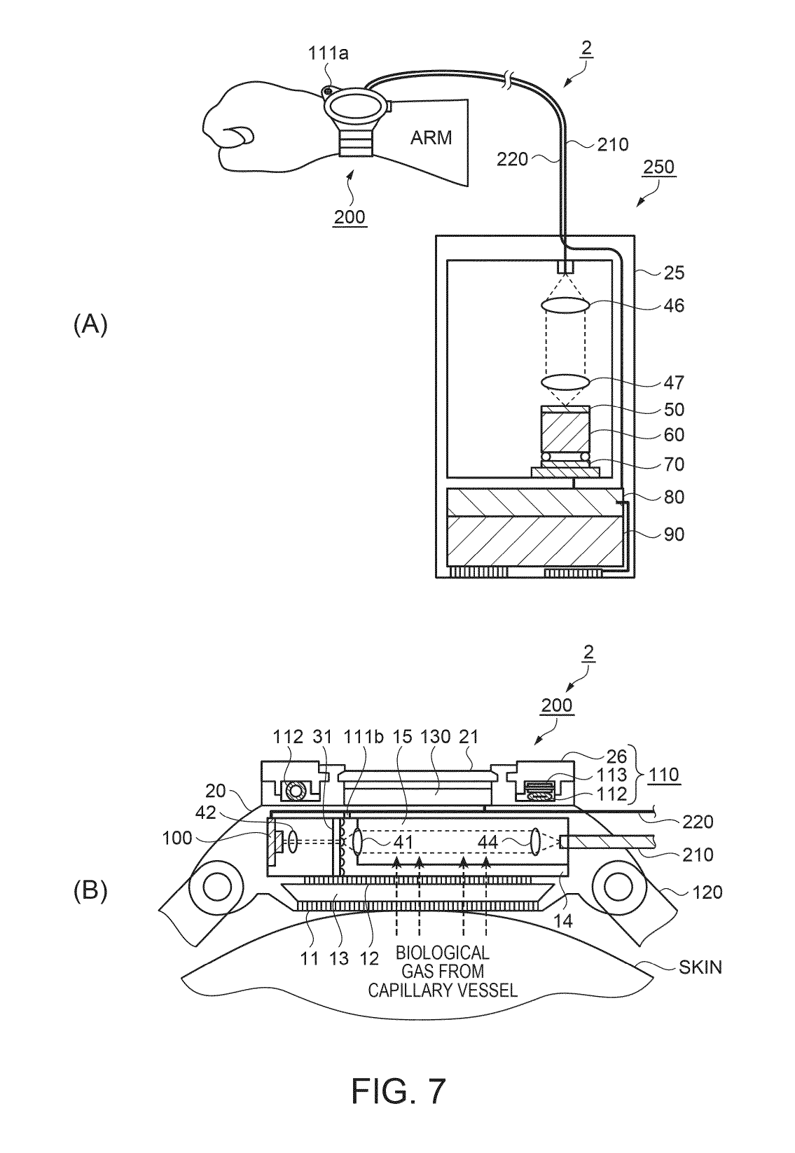

FIG. 7 shows the substance detection device 2 according to the second embodiment, wherein (a) is an explanatory view of the overall structure, and (b) is a cross-sectional view of the main body section 200. As shown in FIG. 7(a), the substance detection device 2 is constituted by the main body section 200 and the detection section 250. The main body section 200 and the detection section 250 are connected to each other through the optical fiber 210 which transmits the enhanced Raman scattered light to the detection section 250 and the cable 220 which supplies electric power from the electric power supply section 90 to the main body section and also inputs an electrical signal processed by the detection section 250 to the main body section 200.

The detection section 250 includes, in a main body case 25, lenses 46 and 47 which condense an enhanced Raman scattered light introduced through the optical fiber 210, an optical filter 50 which removes a Rayleigh scattered light from the condensed enhanced Raman scattered light, a spectrometer 60 which breaks down the enhanced Raman scattered light into a spectrum, a light receiving element 70 which converts the dispersed spectrum to an electrical signal, a signal processing and control circuit section 80 which converts the dispersed spectrum to an electrical signal as information of a fingerprint spectrum specific to acetone detected from the biological gas, and an electric power supply section 90.

A case where the main body section 200 is attached to a wrist part is shown as an example. As shown in FIG. 7 (b), the main body section 200 includes a first permeable membrane 11 which comes in close contact with the human skin, and a second permeable membrane 12 which is placed with a space 13 from the first permeable membrane 11. The first permeable membrane 11 and the second permeable membrane 12 have the same function as that in the above-described first embodiment (see FIG. 1 (b)).

The first permeable membrane 11 and the second permeable membrane 12 are placed on the human body side of the case 20 such that the first permeable membrane 11 comes in close contact with the skin by an attachment belt 120.

On the inner side of the second permeable membrane 12, a sensor chamber 14 and a detection chamber 15 are provided so as to be separated from each other with a partition. The sensor chamber 14 is a space in which a biological gas released from the arm (skin) is stored, and a sensor section 31 (sensor chip 32) is placed therein. The structure and function of the sensor section 31 (sensor chip 32) are the same as those in the first embodiment (see FIG. 4).

In the sensor chamber 14, a light source 100 which excites a molecule to be detected, a lens 42 which condenses a light emitted from the light source 100 on the sensor section 31, and the sensor chip 32 which enhances a Raman scattered light are placed. To the detection chamber 15, the lenses 41 and 42 which condense an enhanced Raman scattered light to be scattered from the sensor chip 32, and the optical fiber 210 which transmits the enhanced Raman scattered light to the detection section 250 are connected. In the sensor chamber 14, an air intake port 111b which communicates with a collected sample discharge unit 110 which discharges the biological gas taken in the sensor chamber 14 to the outside is opened.

As the collected sample discharge unit 110, a tube pump can be used in this embodiment. The tube pump is constituted by a discharge tube 112 with elasticity, a plurality of rotating rollers 113 which press the discharge tube 112, and a rotating ring 26 which moves the position of the rotating rollers 113 from the sensor chamber 14 side to the discharge port 111a side. One end of the discharge tube 112 is the air intake port 111b which communicates with the sensor chamber 14. The rotation of the rotating ring 26 may be performed by hand or driven by a motor.

Further, the light source 100 is connected to the electric power supply section 90 through the optical fiber 210, and electric power is supplied thereto. The display section 130 is connected to the signal processing and control circuit section 80 through the cable 220, and a display signal is input thereto. Further, by inputting an input signal of an operation section 22 to the signal processing and control circuit section 80 through the cable 220, the fat burning measurement is started or ended. Accordingly, the cable 220 is a multilayer or multiaxial cable.

Incidentally, in the case where the display section 130 is an electrooptical display device such as a liquid crystal display device or an organic EL device, a display driver is provided.

On the upper part of the main body section 200 in the drawing, the display section 130 is placed, and on the upper part of the display section 130, a windshield glass 21 is placed and protects the display section 130.

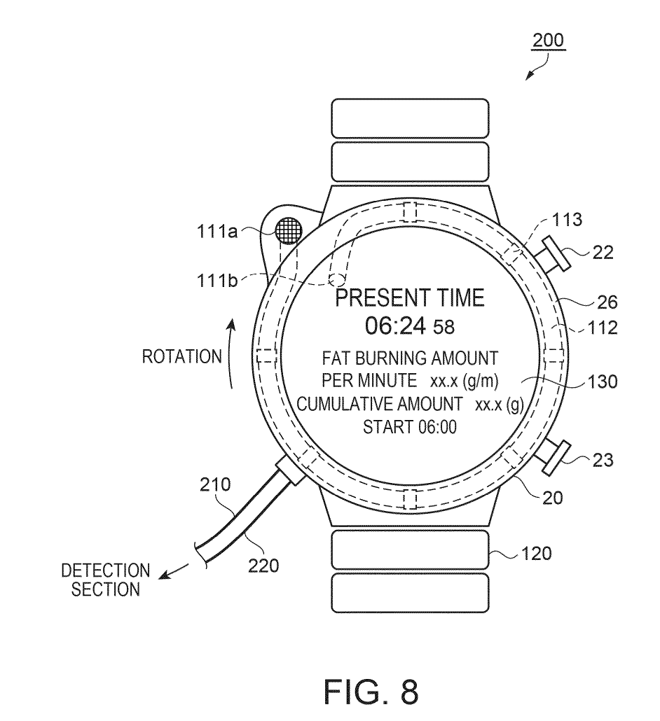

Next, an external plan view of the main body section 200 is illustrated in FIG. 8 and described.

FIG. 8 is an external plan view of the main body section 200 according to this embodiment. Operation sections 22 and 23 for operating the substance detection device 2, the rotating ring 26 which operates the collected sample discharge unit 110 (tube pump) for discharging the biological gas in the sensor section 31, the discharge port 111a for allowing one end of the tube pump to communicate with the outside air, and the like are provided in the case 20. In a central portion, the display section 130 is placed, and a present time, a measurement start time of fat burning, the amount of fat burning (g/m) per unit time (1 minute), the cumulative (integrated) amount of fat burning (g) from the start of measurement, and the like can be displayed. The case 20 is provided with an attachment belt 120 for attaching the device to the arm.

A user first rotates the rotating ring 26 to move the position of the rotating roller 113, and discharges the biological gas in the sensor chamber 14. Subsequently, by pressing the operation section 22, the measurement is started. Then, the display of the measurement start time of fat burning is reset, and the time when the operation section 22 is pressed is displayed, and the measurement of the amount of fat burning is started. By performing appropriate exercise, fat can be burned more than in a resting state. The results are displayed as the amount of fat burning (g/m) per minute and the cumulative amount of fat burning. When the measurement is completed, by pressing the operation section 22, the measurement of fat burning is ended. Incidentally, it is more preferred that in the display section 130, whether the measurement of fat burning can be started (whether the biological gas is discharged from the sensor chamber 14), or an icon for discharging the biological gas is displayed. Further, it is desirable to also display the information of the voltage of the electric power supply section 90.

The substance detection device 2 of this embodiment is divided into the main body section 200 and the detection section 250. As described above, the main body section 200 has a form wearable on the arm (wrist part), and the detection section, which is the other part of the device, can be attached to a place (an arm part, a chest part, an abdominal part, a leg part, or the like) where the detection section hardly becomes an obstacle in daily life and during exercise with a belt (not shown) or the like in a state where it is connected to the main body section 200 through the optical fiber 210 and the cable 220.

According to this configuration, the device is divided into the main body section 200 and the detection section 250, and therefore, each section enables further reduction in size and weight as compared with the integrated device, and for example, the main body section 200 can be attached to a wrist part where the display is easily visually recognized by the test subject oneself, and the detection section 250 can be attached to an arbitrary place where the amount of exercise is small.

### Third Embodiment

Next, a substance detection device 3 according to a third embodiment will be described. The substance detection device according to the second embodiment described above is constituted by the main body section 200 and the detection section 250, and the detection sample collection section 10 of the main body section 200 comes in close contact with the skin such as a wrist part, and thereby the biological gas is directly taken in the sensor chamber 14. On the other hand, the third embodiment has a characteristic that a biological gas collection section is separated from a main body section 202. Therefore, different points from the second embodiment will be mainly described by providing the common components with the same reference numerals as in the second embodiment (see FIG. 7).

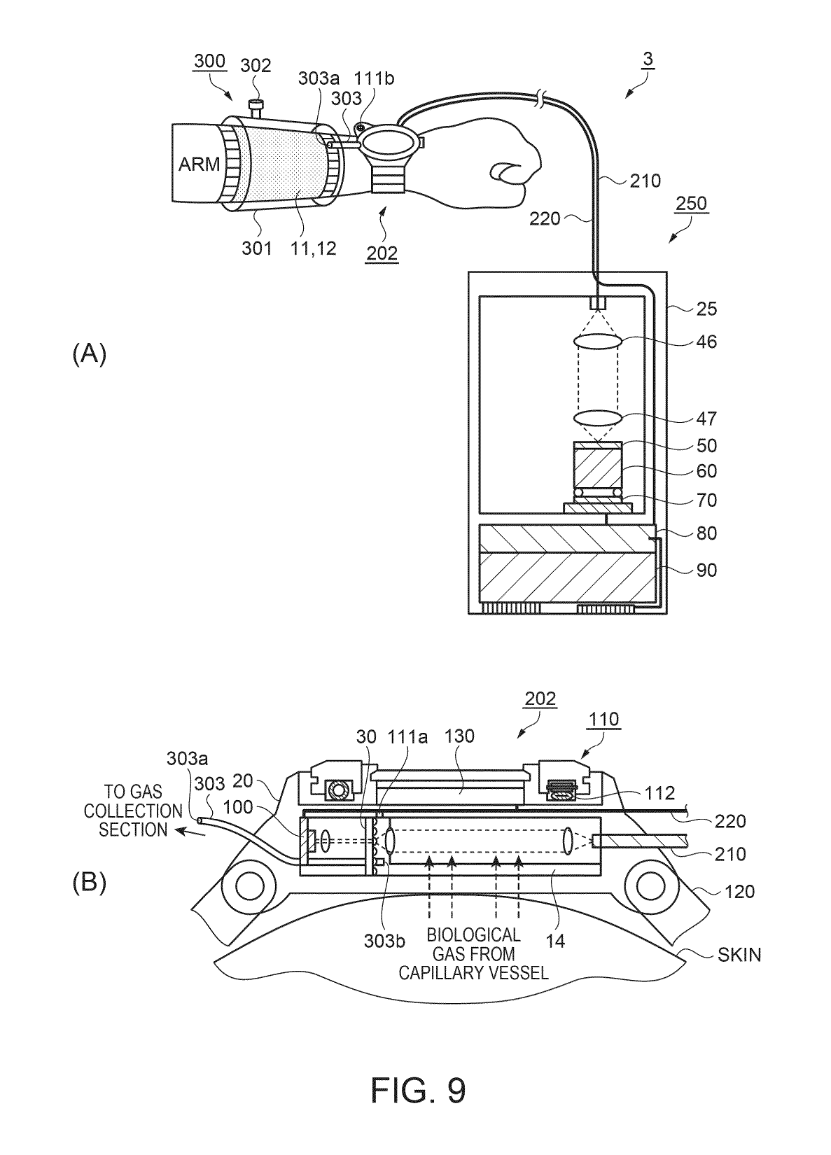

FIG. 9 shows the substance detection device 3 according to the third embodiment, wherein (a) is an explanatory view of the overall structure, and (b) is a cross-sectional view showing the main body section 202.

As shown in FIG. 9(a), the substance detection device 3 is constituted by the main body section 202, a detection sample collection section 300, and a detection section 250. The detection section 250 has the same structure as that in the second embodiment described above. The main body section 202 has substantially the same structure as the detection sample collection section 10 in the second embodiment (see FIG. 7(b)), however, a first permeable membrane 11 and a second permeable membrane 12 are provided in the detection sample collection section 300 with a space provided therebetween. A sensor chamber 14 of the main body section 202 and the detection sample collection section 300 are allowed to communicate with each other through a biological gas introduction tube 303. Incidentally, the detection sample collection section 300 is exaggeratedly shown in the drawing.

The detection sample collection section 300 is placed at a site as close as possible to the main body section 202. In this embodiment, the main body section 202 is attached to the wrist, and the detection sample collection section 300 is attached to an arm portion on the upper part of a wrist part. The detection sample collection section 300 covers the circumference of the arm part with a balloon-shaped external partition 301, and thereby the biological gas can be stored therein. Then, the detection sample collection section 300 is allowed to communicate with the biological gas introduction tube 303 at a site in the vicinity of the main body section 202. Incidentally, between a space surrounded by the external partition 301 and an opening 303a at the end of the biological gas introduction tube 303, a first permeable membrane 11 and a second permeable membrane 12 are provided. The first permeable membrane 11 may be configured to come in close contact with the surface of the arm. part. It is also possible to omit the second permeable membrane 12. An opening 303b at the other end of the biological gas introduction tube 303 communicates with the sensor chamber 14 and introduces the biological gas in the detection sample collection section 300 into the sensor chamber 14.

The external partition 301 is provided with a valve 302, and after measuring the amount of fat burning, the valve 302 is opened to discharge the biological gas in the detection sample collection section 300, and before starting the measurement of the amount of fat burning, the valve 302 is closed, and the biological gas is collected therein.

The biological gas in the sensor chamber 14 is discharged to the outside by a collected sample discharge unit 110 in the same manner as in the second embodiment.

In this embodiment, the detection sample collection section 300 is separated from the main body section 202. According to this configuration, by attaching the main body section 202 to a wrist part, and attaching the detection sample collection section 300 to an arm part in the vicinity of the main body section 202, an area from which the biological gas is collected by the detection sample collection section 300 can be increased, and thus, the collection amount of the biological gas can be increased.

### Fourth Embodiment

Next, a substance detection device 4 according to a fourth embodiment will be described. The substance detection device 1 according to the first embodiment described above has a structure in which the detection sample collection section 10, the detection section 30, and the display section 130 are integrated. On the other hand, the fourth embodiment has a characteristic that the device has a structure in which only a display section 410 is separated from a main body section of the detection device.

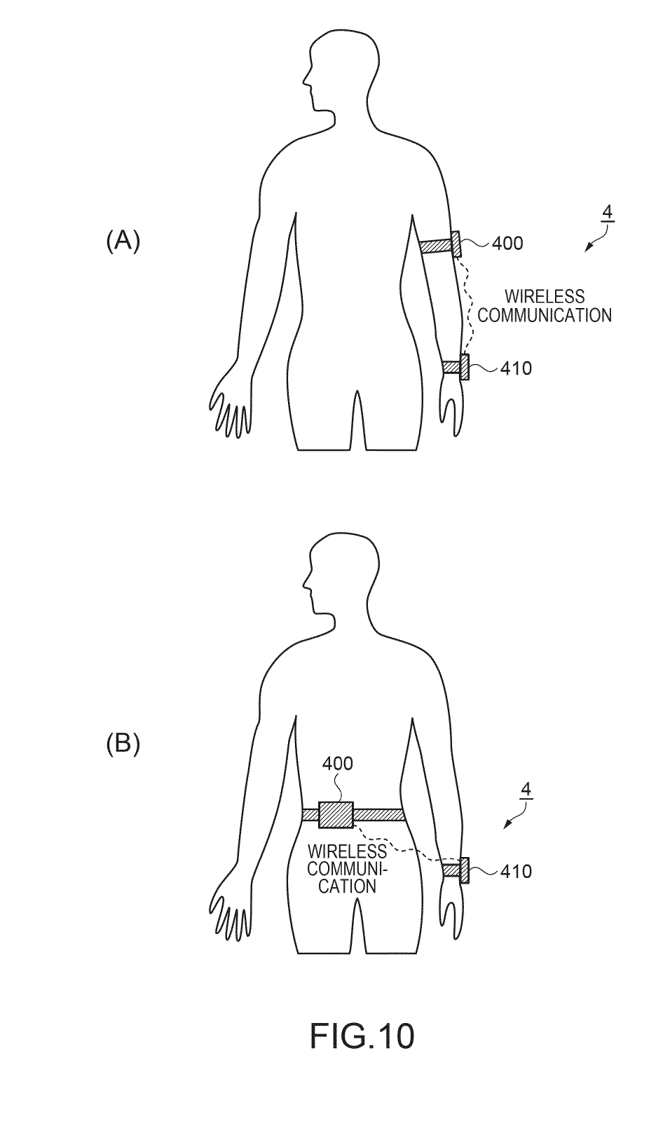

FIG. 10 is an explanatory view of a structure of the substance detection device 4 according to the fourth embodiment, wherein (a) shows a case where a detection device main body section 400 is attached to an arm part, and (b) shows a case where the detection device main body section 400 is attached to an abdominal part. The substance detection device 4 is constituted by the detection device main body section 400 and the display section 410. The detection device main body section 400 is constituted by a detection sample collection section 10 and a detection section 30, and has a structure in which the display section 130 is removed from the substance detection device according to the first embodiment (see FIGS. 1(a) and 1(b)). Therefore, the detection device main body section 400 can be attached to an arbitrary place of the body capable of taking in the biological gas because it does not need to have a visually recognizable structure. Accordingly, the detection device main body section 400 and the display section 410 are provided with an antenna or a wireless communication circuit.

The display section 410 uses an electrooptical display unit such as a liquid crystal display device or an organic EL device, and is stored in a case and is attached to a place of the body where it is easily visually recognized with an attachment belt or the like.

According to such a configuration, the display section 410 may be placed at a site apart from the body, and it is possible to transmit data detected by the detection device main body section 400 to, for example, a PC, a cellular phone, a tablet information device, or the like, and to display the detection result in the display section of such a device. Therefore, the detection result can be recognized at a site apart from the test subject, and by utilizing the memory of a PC or a cellular phone, the previous detection results and the cumulative values over a long period of time can be known.

Incidentally, as the communication unit, not only a wireless communication unit, but also a configuration of connection with a cable and application of optical communication can be adopted.

Incidentally, it is possible to control an appropriate exercise intensity by utilizing the detection of the amount of fat burning, which will be described.

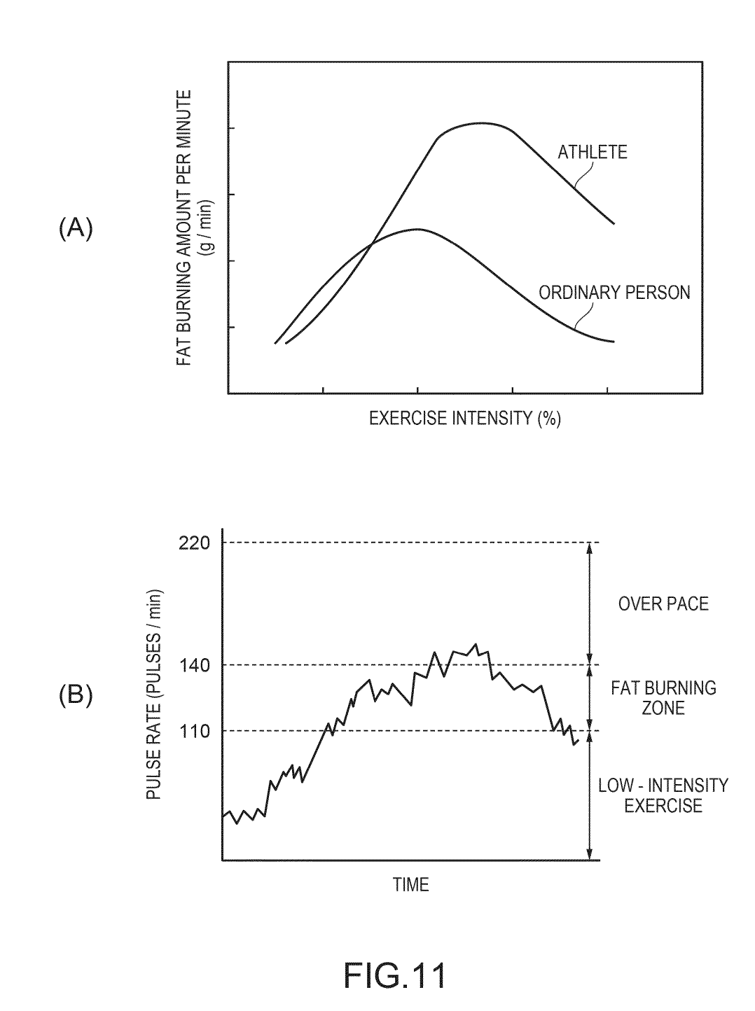

FIG. 11 shows relationships among an exercise intensity, a pulse rate, and the amount of fat burning, wherein (a) is a graph showing a relationship between an exercise intensity and the amount of fat burning, and (b) is a graph showing a relationship between a pulse rate and the amount of fat burning.

As shown in FIG. 11(a), the conditions for obtaining the maximum fat burning ratio (the maximum amount of fat burning per unit time) vary depending on the gender, age, exercise habit, and so on. In the case of an ordinary person, when the exercise intensity is about 40%, and in the case of an athlete, when the exercise intensity is about 50%, the maximum fat burning ratio is obtained. Therefore, in order to efficiently burn fat, it is necessary to appropriately control the exercise intensity individually. That is, the exercise intensity when the maximum fat burning ratio is obtained has been measured in advance individually, and exercise may be performed at the exercise intensity according to an instruction expressed as a numerical value capable of easily performing control also during exercise such as a heart rate or a pulse rate.

The exercise intensity when the maximum fat burning ratio is obtained varies depending on the exercise habit and age individually, and by regularly measuring the exercise intensity when the maximum fat burning ratio is obtained, the effect of fat burning is increased.

For example, in the case shown in FIG. 11(b), a person who has a pulse rate of 110 or less at low-intensity exercise, a pulse rate in the range of 110 to 140 in a fat burning zone, a pulse rate of 140 or more at an over pace can enhance the fat burning efficiency at an exercise intensity at which the pulse rate is in the range of 110 to 140. Based on this, if exercise is performed according to an appropriate exercise intensity for a given time, and the effect of the exercise can be confirmed, the motivation to continue exercise is increased, and as a result, a continuous effect can be expected.

The respective substance detection devices described in the above embodiments can measure the amount of fat burning during exercise, and has a characteristic that by selecting an appropriate exercise intensity at which the maximum fat burning ratio is obtained and performing exercise at the appropriate exercise intensity, efficient fat burning can be realized, and also can be confirmed by oneself.

### REFERENCE SINGS LIST

1: substance detection device, 10: detection sample collection section, 11: first permeable membrane, 12: second permeable membrane, 14: sensor chamber, 31: sensor section, 60: spectrometer, 70: light receiving element, 80: signal processing and control circuit section, 90: electric power supply section, 100: light source, 130: display section

The entire disclosure of Japanese Patent Application No. 2012-146529, filed Jun. 29, 2012 is expressly incorporated by reference herein.

1. A substance detection device, comprising:
a detection sample collection section which collects a biological gas released from the human skin, and stores the gas in a sensor chamber;
a light source which excites a Raman scattered light from a substance to be detected in the collected biological gas;
a sensor section which enhances the Raman scattered light by localized surface plasmon resonance;
a spectrometer which disperses the enhanced Raman scattered light;
a light receiving element which converts the dispersed light to an electrical signal and acquires the spectrum of the enhanced Raman scattered light;
a signal processing and control circuit section which compares the acquired spectrum with the fingerprint spectrum of the substance to be detected which has been stored in advance and thereby identifies the substance to be detected, and calculates the concentration of the substance to be detected and the amount of a specific substance having a correlation with the concentration of the substance to be detected; and
a display section which displays the results calculated by the signal processing and control circuit, wherein
the detection sample collection section comes in close contact with the human skin, and the detection sample collection section includes a permeable membrane which allows the biological gas to pass through the sensor section.

2. The substance detection device according to claim 1, wherein the sensor section includes a sensor chip having a metal nanostructure which is smaller than the wavelength of a light emitted from the light source.

3. The substance detection device according to claim 1, further comprising a collected gas discharge unit which discharges the biological gas stored in the sensor chamber outside the sensor chamber.

4. The substance detection device according to claim 1, wherein the detection sample collection section, the light source, the sensor section, the spectrometer, the light receiving element, the signal processing and control circuit section, and the display section are integrated so as to be wearable on the body.

5. The substance detection device according to claim 1, wherein the substance detection device is divided into
a main body section in which the detection sample collection section, the light source, the sensor section, and the display section are integrally housed, and
a detection section in which the spectrometer, the light receiving element, and the signal processing and control circuit section are integrally housed, and
the main body section and the detection section are connected to each other through an optical fiber which transmits the enhanced Raman scattered light and a cable which supplies electric power and transmits an electrical signal.

6. The substance detection device according to claim 1, wherein
the detection sample collection section is separated from a main body section in which the light source, the sensor section, and the display section are integrally housed, and
the detection sample collection section and the sensor chamber are allowed to communicate with each other through a biological gas introduction tube.

7. The substance detection device according to claim 1, wherein
the substance detection device includes a display section which is separated from a detection device main body section in which the detection sample collection section, the light source, the sensor section, the spectrometer, the light receiving element, and the signal processing and control circuit section are integrally housed, and
the detection device main body section and the display section are connected to each other through a communication unit.

8. The substance detection device according to claim 1, wherein
the substance to be detected is acetone and the specific substance is body fat,
the signal processing and control circuit section calculates the burning amount of the body fat based on the amount of the detected acetone, and
the display section displays the burning amount of the body fat.

9. A body fat burning measurement device, comprising:
a display section provided on the outer surface of a watch-type case;
a sensor section which detects a target substance in a biological gas released from a test subject by applying plasmon resonance;
a light source section which irradiates the sensor section with a laser light to excite a Raman scattered light;
a control section which calculates body fat burning according to the detected concentration of the target substance, and displays the calculation result in the display section;
a close contact section which includes a permeable membrane for allowing the biological gas to pass therethrough and is capable of coming in close contact with a part of the arm of the test subject; and
a wrist band which enables the close contact section to be attached to the arm of the test subject, wherein
the display surface, the emitting direction of the laser light, and the permeable membrane are disposed parallel to one another.
